(19) United States
(12) Patent Application Publication
(10) Pub. No.: US 2025/0381121 A1
(43) Pub. Date: Dec. 18, 2025

(54) COMPOSITION FOR PERSONAL CARE PRODUCTS

(71) Applicant: VAGINAL BIOME SCIENCE, INC., Portland, OR (US)

(72) Inventors: Wendy STRGAR, Portland, OR (US); Kimberly CAPONE, Portland, OR (US); Euen GUNN, Hopewell, NJ (US)

(21) Appl. No.: 19/240,577

(22) Filed: Jun. 17, 2025

Related U.S. Application Data
(60) Provisional application No. 63/660,769, filed on Jun. 17, 2024.

(51) Int. Cl. A61K 8/73; A61K 8/34; A61K 8/365; A61K 8/46; A61Q 19/00

(52) U.S. Cl. CPC A61K 8/73; A61K 8/342; A61K 8/365; A61K 8/466; A61Q 19/00

(57) ABSTRACT

A composition for personal care products comprises succinoglycan, brassicyl valinate esylate, cetearyl alcohol, and optionally acemannan. Another composition for personal care products comprises acemannan and has a prebiotic effect for vaginal bacteria. The composition for personal care products has suitable viscosity to be used as a base of personal care products such as a cream, a lotion, a lubricant, an ointment, a syrup, a gel, or the like and does not include harmful components. The composition for personal care products is stable under various pH, including acidic, neutral, and basic pH, and the viscosity does not deteriorate over storage. The composition for personal care products is compatible with wide ranges of salt and electrolyte concentrations, pH, and osmolality found in human body.

### CROSS-REFERENCE TO RELATED APPLICATION

This application claims the benefit of U.S. Provisional Application No. 63/660,769, filed Jun. 17, 2024, the disclosure of which is hereby incorporated by reference in its entirety.

### FIELD

This disclosure relates to a composition for personal care products. More specifically, this disclosure relates to a composition for personal care products that has viscosity suitable for a cream, a lotion, a lubricant, an ointment, a syrup, a gel, or the like and is stable in acidic pH. An example of personal care products include vaginal care products.

### BACKGROUND OF THE INVENTION

Personal care products include active ingredients and inactive ingredients. Inactive ingredients are substances included in a product that generally do not provide direct health or care benefits but be a base for the product, providing support to product's formulation, stability, and usability. Properties of personal care products such as viscosity and texture are generally adjusted by inactive ingredients to be suitable for the intended use. As personal care products can come into contact with the subject using the products, it is desired that the amounts of irritable components in the personal care products are minimized. Some base compositions used in personal care products are not stable in low pH and are not suited to support low-pH ingredients.

A healthy vagina has a unique fluid composition and low pH, which is different from other parts of the body. A healthy vaginal microbiome is dominated by several species of Lactobacillus, which produce lactic acid. The lactic acid produced by these bacteria results in vaginal fluids having an acidic pH, in the range of 3.5 to 4.5. A healthy vaginal environment supporting a healthy vaginal microbiome has salts (ions), sugars, and other osmotically active solutes present in suitable amounts to be osmotically balanced. Disruption of vaginal pH, osmolality, and/or solute composition may kill beneficial bacteria and may lead to a dysbiotic condition. Thus, it is desired that topical vaginal products are matched to the natural fluid composition of the vagina. However, some vaginal products are prepared with different pH, osmolality, or salt concentrations and ratios from the natural vaginal fluid.

### SUMMARY OF THE INVENTION

This disclosure relates to a composition for personal care products. More specifically, this disclosure relates to a composition for personal care products that has viscosity suitable for a cream, a lotion, a lubricant, an ointment, a syrup, a gel, or the like and is stable in any pH, including acidic, neutral, and basic pH. An example of personal care products include vaginal care products.

In an embodiment, a composition for personal care products comprises succinoglycan, brassicyl valinate esylate, and cetearyl alcohol. The composition for personal care products has suitable viscosity to be used for a personal care product. The composition for personal care products does not include harmful components and is stable over time under various pH and salt ranges.

In an embodiment, a composition for personal care products comprises succinoglycan, brassicyl valinate esylate, cetearyl alcohol and acemannan. The composition for personal care products has suitable viscosity to be used for a personal care product. The composition for personal care products does not include harmful components and is stable over time under various pH and salt ranges.

In an embodiment a composition for personal care products comprises acemannan and has prebiotic effect for vaginal bacteria. The composition for personal care products has suitable viscosity to be used as a personal care product. The composition for personal care products does not include harmful components and is stable over time under various pH and salt ranges.

### BRIEF DESCRIPTION OF THE DRAWINGS

FIG. 1 is a flowchart depicting a method of bio-matching a composition for personal care products to a living body.

FIG. 2 shows concentrations of ions in vaginal fluid in mOsm/kg.

FIG. 3 shows ratios of ions in vaginal fluid.

FIG. 4 shows ranges of pH in vaginal fluid for healthy or disease conditions, and for some embodiments.

FIG. 5 shows ranges of ion concentrations in % w/v in embodiments.

FIG. 6 shows ratios of ions in vaginal fluid in embodiments.

FIG. 7 shows possible ranges of ion concentrations in % w/v in embodiments.

FIG. 8 shows possible ranges of ion concentrations in % w/v in embodiments.

FIG. 9 shows potassium, sodium, and calcium concentrations in blood, fluid extracted from brain and cardiac tissue, and in vaginal fluid.

FIG. 10 is a graph showing the growth of Lactobacillus crispatus in a culture with various concentrations of acemannan or without acemannan.

### DETAILED DESCRIPTION OF THE INVENTION

This disclosure relates to a composition for personal care products. More specifically, this disclosure relates to a composition for personal care products that has viscosity suitable for a cream, a lotion, a lubricant, an ointment, a syrup, a gel, or the like and is stable in any pH, including acidic, neutral, and basic pH. An example of personal care products include vaginal care products.

### Definitions

An “ion” is a charged particle that is dissolved in water or another solvent. Ions are also commonly called “electrolytes.” Ions may consist of a single atom as in the case of sodium or chloride or may be polyatomic as in the case of bicarbonate or benzoate. Ions may be positively charged (“cations”), such as sodium, potassium, calcium, or magnesium; or they may be negatively charged (“anions”), such as chloride, bicarbonate, sorbate, or benzoate. When a cation and an anion crystallize (come out of solution to form a solid), together they can form a “salt,” such as sodium chloride, potassium benzoate, potassium sorbate, or sodium bicarbonate, for example.

“Osmolality” and “osmolarity” are defined as measures of how a dissolved substance (“solute”) affects the movement of water. Water molecules tend to distribute themselves with respect to ions or other osmotically active molecules, with water moving into areas with high concentrations of solutes and away from areas with low concentrations of solutes. Additionally, water molecules can interact with charged areas on other kinds of large, complex molecules; molecules with multiple charged areas can ‘draw’ water molecules in a way that molecules without charged areas do not. The osmolality of a product, therefore, can be dependent upon both the concentration/quantity of molecules in a product and the specific types of osmotically active solutes in the product.

“Osmolality” is defined as osmoles of a substance per kilogram of water, while “osmolarity” is defined as osmoles of a substance per liter of water. Since a liter of pure water has a mass of one kilogram, osmolarity and osmolality herein refer to identical concepts. Osmolality can be measured by any osmometer, for example, an osmometer (Model 3320) from Advanced Instruments Inc.

“Hyperosmolal,” “hyperosmolar,” or “hypertonic” products are defined herein as those with osmolality higher than the osmolality of vaginal epithelial cells (approx. 290-450 mOsm/kg). Hypertonic products would have an osmolality higher than approximately 500 mOsm/kg. When these products are applied to living cells, water can move out of the cell into the product. This can cause the cells to shrink and quickly die. This effect can underlie the damaging effects of hyperosmolar or hypertonic vaginal products.

“Iso-osmolal,” “iso-osmolar,” or “isotonic” products are defined as those with osmolality similar to that of the contents of living cells, approx. 290-450 mOsm/kg. Consequently, when an iso-osmolal product is applied to living cells, there is no net movement of water either into or out of the cells. Product components which ensure the products are isotonic to vaginal secretions include sodium salts, potassium salts, calcium salts, magnesium salts, or manganese salts.

“Hypo-osmolal,” “hypo-osmolar,” or “hypotonic” products are defined herein as those with osmolality lower than vaginal epithelial cells. Hypotonic products would have an osmolality of less than 200 mOsm/kg. Consequently, when the product is applied to living cells, water moves from the product into the cells. This can cause the cells to swell and burst, in theory. In practice, however, it is difficult to formulate products to be hypotonic, and living cells are quite good at protecting themselves against hypotonic stresses.

A “healthy vaginal microbiome” is defined as one with high levels of beneficial bacteria and low levels, or no levels, of pathogenic bacteria, resulting in a vaginal pH between 3.6 and 4.2 or 3.5 to 4.5. Often, but not always, a healthy vaginal microbiome is dominated by one or more Lactobacillus species, including L. crispatus, L. salivarius, L. rhamnosus, L. acidophilus, L. plantarum, L. gasseri, and L. fermentum, among others. Unhealthy vaginal microbiomes may have elevated levels of Gardnerella vaginosis, Atopobium vaginae, and Megasphaera spp., and/or many others; a healthy vaginal microbiome contains low or no levels of these organisms.

“Bio-matching” is the method of formulating a product to have similar components as those found in the part of the body for which the product is intended for use. A method for bio-matching is described, for example, in U.S. Pat. Nos. 9,470,676 and 10,195,169. A product “bio-matched” to the vagina would be isotonic to healthy vaginal secretions. A product bio-matched to the vagina may include sodium, potassium, calcium, and other ions at ratios matching those found in healthy vaginal secretions. A product bio-matched to the vagina may include sodium, potassium, calcium, and other ions at concentrations or amounts matching those found in healthy vaginal secretions. A product bio-matched to the vagina may contain racemic lactic acid consisting of, for example, approximately 50% L and 50% D enantiomers of lactic acid. A product bio-matched to the vagina may include Lactobacillus spp. or other bacteria typically found in a healthy vagina. Because there is a wide range of what is considered healthy in terms of pH, lactic acid concentrations, ion ratios, and microbiome constitution, bio-matching to the within the healthy range is feasible, while attempting to produce bio-identical products for individuals is not.

“Vaginal product” or “vaginal care product” are defined herein as products intended to be applied to vagina, genitals or the like to improve, maintain, or promote condition of the target tissue for purposes including, but not limited to, medical, supplemental, and cosmetic purposes.

“Personal product” or “personal care product” are defined herein as products intended to be applied to subjects including human or other animals to improve, maintain, or promote condition of the subjects, and includes but are not limited to products for medical, supplemental, and cosmetic purposes.

### Problems to be Solved

Portions of a human body host microbial populations which support health of the tissues of the body. Together, the microbes and their environment—the tissue in which they live—constitute a “microbiome.” A healthy microbiome is supported by a healthy environment, including optimal pH, optimal osmolality, and optimal salt concentrations. Healthy microbiomes can provide benefits to the body and/or protection against pathogenic organisms. A microbiome may contain multiple microbes. For example, the gut microbiome hosts a variety of different microbes; some of these produce vitamins for the body, while others prevent overgrowth of yeast or pathogenic bacteria. In another example, the microbiome of the vagina hosts a variety of different microbes, especially Lactobacillus spp., which may beneficially modulate the person's immune system, and protect against infectious organisms.

It is preferred that personal care products applied to human body do not disrupt the healthy microbiome existing in the body parts that the products are to be applied. It is also preferred that personal care products maintain, restore, or promote the healthy microbiome. These properties can be achieved by using a bio-matched formulation.

For example, some measures to assess the health of the vagina are, levels of Lactobacillus, pH, lactic acid levels, moisture levels, vaginal epithelial cell vitality, and levels and ratios of ions, sugars, or other osmotically active solutes. A healthy vaginal microbiome is dominated by several species of Lactobacillus, a genus of Gram-positive facultative anaerobic rod-shaped bacteria which produce lactic acid as a byproduct of metabolizing sugars. The lactic acid produced by these bacteria results in vaginal fluids having a very acidic (low) pH, in the range of 3.5 to 4.2 or 3.5 to 4.5 depending on the exact Lactobacillus species; this is one of the lowest pH levels found in the human body. This acidic environment prevents overgrowth of pathogenic bacteria and promotes health of vaginal and other reproductive tissues.

In addition to optimal pH and lactic acid levels, a healthy vaginal environment supporting a healthy vaginal microbiome must have salts (ion), sugars, and other osmotically active solutes present in the correct amounts to be osmotically balanced. Products which are bio-matched to these parameters are not damaging to the healthy microbiomes and healthy vaginal tissues.

However, many personal care products available on the market are not bio-matched. (e.g., not iso-osmolal with the target tissue, do not have the salt concentration and salt balance similar to the target tissue, or are not at the pH of the target tissue) and can damage the target tissue. Sometimes, personal care products may contain ingredients that are harmful to the tissue.

One of the reasons such unideal products are on the market is the difficulty in developing a base composition (a support, a viscosity modifier, or inactive ingredients etc.) that is compatible with the bio-matched conditions. For example, a molecular network formed in the base composition and generate viscosity may break down under acidic conditions over time. Instead of developing a base composition that tolerates the bio-matched components, many products are prepared, for example, at higher pH, higher osmolality, or use different salt concentrations and salt ratios from the bio-matched condition.

For example, in the case of vaginal lubricants, most lubricants available over the counter such as K-YR JELLY, K-Y® PLUS, GYNOL II®, CONCEPTROL®, WET® ORIGINAL, ASTROGLIDER, ID GLIDER, PREPAIR®, ELBOW GREASE®, REPLENS® AND BOY BUTTER®; and surgical lubricants used in clinic such as SURGILUBER are formulated hyperosmolal than a typical vaginal fluid. In addition, the pH is set significantly higher than that of typical healthy vaginal fluid. When such lubricants are applied to the vagina, they may disrupt the healthy microbiome and damage the vaginal tissue because of the high osmolality. For example, using a hyperosmolal lubricant may lead to increased rates of vaginal epithelial cell death and cell sloughing from the vaginal epithelium because a hyperosmolal environment can induce vaginal cells to release water and die. Given the level of breach in the epithelial barrier by hypertonic lubricants, there is potential of an increase in susceptibility to sexually transmitted infections such as HIV and HSV in individuals who are regular users of hypertonic lubricants.

Using a lubricant with higher pH than the target tissue can also cause damage to the tissue. For example, in the case of vaginal lubricants, products having higher pH than the vaginal fluid may disrupt the healthy microbiomes that help to maintain vaginal acidity. These disruptions may further lead to subsequent dryness of the vagina and increased susceptibility to dysbiosis and infection.

Furthermore, the existing vaginal lubricants can disrupt the concentration of sodium, potassium, and calcium ions in the vaginal fluid if they are not bio-matched and can result in a loss of epithelial cell vitality. A healthy vaginal epithelium normally consists of 30-50 layers of cells with intact intercellular junctions, produces high quantities of glycogen, and has high transepithelial electrical resistance (TEER). The use of personal products that are not bio-matched may cause elevated sodium concentrations and a different sodium to potassium ratio from the normal ranges and may affect the epithelial cell vitality.

Moreover, the existing vaginal products may include ingredients that are harmful to the vagina to control viscosity or to extend the shelf life. For example, many vaginal products typically include detergents and surface-active agents (surfactants), glycerol (glycerin) and other humectant/solvent excipients, and/or preservatives which typically include chlorhexidine and/or ethylenediaminetetraacetic acid (EDTA), among others. Detergents and surface-active ingredients can be harmful because they are toxic or harmful to mucosal epithelia, including that of the vagina. Such detergents and surface-active ingredients may include nonoxynol-9 (N9) and similar detergents, and glycerol monolaurate (GML). Glycerol (glycerin) and other humectant/solvent excipients can be harmful because they increase vaginal susceptibility to disease. For example, Moench et al. (BMC Infectious Diseases 2010, 10:331) reported that the following excipients markedly increased the subject's susceptibility to herpes simplex virus (HSV-2) after a single exposure: 5% glycerol monolaurate (GML) formulated in K-Y® Warming Jelly, 5% GML as a colloidal suspension in phosphate buffered saline, K-Y® Warming Jelly alone, and both of the humectant/solvent ingredients (neat propylene glycol and neat polyethylene glycol (PEG-8).

Thus, using a general-purpose lubricant such as SURGILUBE® or lubricants that are not bio-matched, such as K-Y® jelly, for vaginal lubrication is not ideal for vaginal health because they may disrupt the unique vaginal fluid composition and the microbiome. In order not to disrupt the healthy vaginal microbiome, a bio-matched lubricant that has pH and osmolality in the range of a healthy vagina (pH in the range of 3.5-4.5, osmolality in the range of 200 to 450 mOsm/kg, and salts at the appropriate concentrations and ratios) is desired. In addition, a lubricant without harmful components is desired. However, there is a problem that the existing lubricant formulations are not stable at low pH. When the pH, osmolality and salts were adjusted to match the vaginal fluid, the components may break down or the lubricant does not maintain consistency upon storage.

### Solutions to the Problems

### Composition for Personal Care Products

The present invention solves the problem that existing compositions for personal care products may not be stable in certain pH, for example in an acidic pH. In particular, the present invention provides a composition for vaginal care products that has suitable viscosity and does not disintegrate when the components are bio-matched to the vaginal fluid and is safer to use for vagina. Although the present specification will describe the composition for personal care products using vaginal care products as an example, it is appreciated that the application of the composition for personal care products is not limited to vaginal use and can be adapted for use for other body parts, as the composition for personal care products can be prepared with wide range of pH and osmolality and remain stable over storage. The composition for personal care products can be used to prepare a safer alternative to the currently available lubricants such as K-Y® jelly and SURGILUBE®.

A composition A for personal care products comprising succinoglycan, brassicyl valinate esylate, and cetearyl alcohol is provided.

The inventors have diligently searched for substitute materials for currently available thickeners and unexpectedly found that when succinoglycan, brassicyl valinate esylate, and cetearyl alcohol are mixed at a certain ratio, a stable gel with high viscosity is formed. The formed gel does not disintegrate after long-term storage in a sealed container at any pH, including acidic, neutral, or basic pH. The composition for personal care products comprising succinoglycan, brassicyl valinate esylate, and cetearyl alcohol (composition A) is described in detail below.

Embodiments of the composition A for personal care products of the present application may be provided in the form of a gel, a lubricant, a suppository, or other suitable forms for vaginal application, or combinations thereof. The composition A for personal care products contains, relative to 100% of the total weight of the composition, succinoglycan at 4% or lower, for example, at 0.1 to 4%, and the combined amount of brassicyl valinate esylate and cetearyl alcohol at 6% or lower, for example, at 0.01 to 6%. A preferable embodiment of the composition for personal care products contains succinoglycan at 2% or lower, for example, at 0.1% to 2%, and the combined amount of brassicyl valinate esylate and cetearyl alcohol at 4% or lower, for example, at 0.01 to 4%. A more preferable embodiment contains succinoglycan at 1% or lower, for example, at 0.39% to 1%, and the combined amount of brassicyl valinate esylate and cetearyl alcohol at 0.3% or lower, for example 0.05% to 0.3%.

The succinoglycan can be any succinoglycan molecules, and can be for example, a product identified as CAS-NO. 73667-50-2 (PubChem CID 154586087). The succinoglycan can be a product formulated and sold as ingredients for personal care products, and can be for example, RHEOZAN® available from SYENSQO as rheology modifiers. Examples of RHEOZAN® include RHEOZAN®SH, RHEOZAN® BLC W, and RHEOZAN®BLC.

The brassicyl valinate esylate and cetearyl alcohol can be any brassicyl valinate esylate and cetearyl alcohol molecules and can be a product prepared as a mixture and sold as ingredients for personal care products. The brassicyl valinate esylate and cetearyl alcohol mixture can be, for example AMINOSENSYL™ SC available from INOLEX.

The composition A for personal care products has viscosity at 30000-90000 cps or higher as measured using a NDJ5S rotational viscometer from Shandong Drick Instruments with a #4 spindle at 6 rpm. Preferably the viscosity is 40000-60000 cps.

The composition A for personal care products has osmolality, for example, of 250 to 450 mOsm/kg, from 300 to 400 mOsm/kg, from 300 to 450 mOsm/kg, from 290 to 450 mOsm/kg, from 250 to 500 mOsm/kg, from 340 to 360 mOsm/kg, up to 500 mOsm/kg, from 100 to 500 mOsm/kg, or from 50 to 1000 mOsm/kg. In an embodiment, the composition for personal care products has osmolality of 100 to 500 mOsm/Kg, preferably 250 to 450 mOsm/Kg, and more preferably 340 to 360 mOsm/Kg.

In an embodiment, the composition A for personal care products has pH in the range of 3.6 to 4.2. In some embodiments, the product may have pH suited for the subject to be applied in the range of, for example, but not limited to, 2.5 to 5.5, 2.5 to 8.0, 2.9 to 3.7, 2.9 to 4.6, 3.0 to 5.0, 3.5 to 4.2, 3.5 to 4.5, 3.7 to 4.7, 3.8 to 4.6, 4.0 to 4.5, 4.2 to 4.6, or 4.6 to 5.0.

It is appreciated that the composition A has viscosity suited as a support (an inactive ingredient) of personal care products at any pH, including acidic, neutral, or basic pH, and does not break down or deteriorate and change viscosity over time. For example, the viscosity of the composition does not significantly change after storing at 40° C. for 40 days. For example, the change rate of the viscosity of the composition after storing at 40° C. for 40 days is less than 10%. Accordingly, the composition A can be combined with ingredients at various conditions, as discussed below.

A composition B for personal care products comprising succinoglycan, brassicyl valinate esylate, cetearyl alcohol, and acemannan is provided.

The composition B comprises essentially the same components at the same amounts as the composition A described above, and further contains, relative to 100% of the total weight of the composition, acemannan at 2% or lower, preferably 1% or lower, for example, at 0.1 to 1.0%, and more preferably at 0.2 to 0.5%.

Acemannan is a D-isomer mucopolysaccharide in aloe vera leaves and can be any preparation of acemannan identified by the CAS NO. 110042-95-0. Acemannan can be a powder prepared and sold as ingredients of personal care products. Acemannan can be from commercial sources, for example, Naturaloe. Acemannan can be an UV-irradiated acemannan.

The composition B for personal care products has viscosity at 30000-90000 cps or higher as measured using a NDJ5S rotational viscometer from Shandong Drick Instruments with a #4 spindle at 6 rpm. Preferably the viscosity is 40000-60000 cps.

The composition B for personal care products has osmolality, for example, of 250 to 450 mOsm/kg, from 300 to 400 mOsm/kg, from 300 to 450 mOsm/kg, from 290 to 450 mOsm/kg, from 250 to 500 mOsm/kg, from 340 to 360 mOsm/kg, up to 500 mOsm/kg, from 100 to 500 mOsm/kg, or from 50 to 1000 mOsm/kg. In an embodiment, the composition for personal care products has osmolality of 100 to 500 mOsm/Kg, preferably 250 to 450 mOsm/Kg, and more preferably 340 to 360 mOsm/Kg.

In an embodiment, the composition B for personal care products has pH in the range of 3.6 to 4.2. In some embodiments, the product may have pH suited for the subject to be applied in the range of, for example, but not limited to, 2.5 to 5.5, 2.5 to 8.0, 2.9 to 3.7, 2.9 to 4.6, 3.0 to 5.0, 3.5 to 4.2, 3.5 to 4.5, 3.7 to 4.7, 3.8 to 4.6, 4.0 to 4.5, 4.2 to 4.6, or 4.6 to 5.0.

It is appreciated that the composition B has viscosity suited as an inactive ingredient of personal care products at any pH, including acidic, neutral, or basic pH, and does not break down or deteriorate and change viscosity over time. For example, the viscosity of the composition does not significantly change after storing at 40° C. for 40 days. For example, the change rate of the viscosity of the composition after storing at 40° C. for 40 days is less than 10%. Accordingly, the composition B can be combined with ingredients at various conditions, as discussed below.

A composition C for personal care products comprising acemannan is provided. The composition C contains acemannan at 2% or lower, preferably 1% or lower, for example, at 0.1 to 1.0%, and more preferably at 0.2 to 0.5% and has a prebiotic effect for vaginal bacteria. The acemannan is as described above and can be an UV-irradiated acemannan. The vaginal bacteria may be Lactobacilli, and the species of Lactobacilli can be Lactobacillus crispatus.

The composition C for personal care products has viscosity at 30000-90000 cps or higher as measured using a NDJ5S rotational viscometer from Shandong Drick Instruments with a #4 spindle at 6 rpm. Preferably the viscosity is 40000-60000 cps.

The composition C for personal care products has osmolality, for example, of 250 to 450 mOsm/kg, from 300 to 400 mOsm/kg, from 300 to 450 mOsm/kg, from 290 to 450 mOsm/kg, from 250 to 500 mOsm/kg, from 340 to 360 mOsm/kg, up to 500 mOsm/kg, from 100 to 500 mOsm/kg, or from 50 to 1000 mOsm/kg. In an embodiment, the composition for personal care products has osmolality of 100 to 500 mOsm/Kg, preferably 250 to 450 mOsm/Kg, and more preferably 340 to 360 mOsm/Kg.

In an embodiment, the composition C for personal care products has pH in the range of 3.6 to 4.2. In some embodiments, the product may have pH suited for the subject to be applied in the range of, for example, but not limited to, 2.5 to 5.5, 2.5 to 8.0, 2.9 to 3.7, 2.9 to 4.6, 3.0 to 5.0, 3.5 to 4.2, 3.5 to 4.5, 3.7 to 4.7, 3.8 to 4.6, 4.0 to 4.5, 4.2 to 4.6, or 4.6 to 5.0.

It is appreciated that the composition C has viscosity suited as an inactive ingredient of personal care products at any pH, including acidic, neutral, or basic pH, and does not break down or deteriorate and change viscosity over time. For example, the viscosity of the composition does not significantly change after storing at 40° C. for 40 days. For example, the change rate of the viscosity of the composition after storing at 40° C. for 40 days is less than 10%. Accordingly, the composition B can be combined with ingredients at various conditions, as discussed below.

It is appreciated that the composition for personal care products (compositions A, B, or C) can be used as an inactive ingredient/thickener to prepare a personal care product. In the prepared personal care product, the composition for personal care products can function to support component such as, but not limited to, lactic acid, salt, and active ingredients, as discussed below.

In an embodiment, a prepared personal care product may contain, relative to the 100% of the total weight of the composition, lactic acid at 0.1 to 5%, preferably at 1 to 3%, and more preferably at 1.5%. Examples of the amount of lactic acid include, but are not limited to, up to 2%, up to 1.5%, 1-3%, 0.5-1%, 0.5-1.5%, 0.5-2.5%, 0.2-2%, 0.1-0.2%, 0.1-5%, 0.05-2.0%, 0.01-2.5%, 0.01-3.0%, 1.0-3.0%, and about 1%. The lactic acid may have a racemic index, which is represented as a ratio of the L and D forms as L/D, of 0/100, 10/90, 20/80, 30/70, 40/60, 50/50, 60/40, 62.5/37.5, 70/30, 80/20, and 90/10.

In an embodiment, a prepared personal care product may contain a calcium salt, a sodium salt, a potassium salt, and optionally a magnesium salt. In an embodiment, the sodium salt is sodium chloride (NaCl), the calcium salt is calcium chloride (CaCl₂)), the potassium salt is potassium chloride (KCl), and the magnesium salt is magnesium chloride (MgCl₂). The concentration of the salts in the personal care products may be, but not limited to, the following ranges: a potassium salt may be included up to 0.5%, at 0.15-0.35%, 0.01-1.5%, 0.001-1.5%, 0.05-0.5%, 0.1-0.5%, or 0.2-0.3% by weight; a sodium salt may be included up to 1.5%, at 0.3-1.0%, 0.15-0.35%, 0.1-1.5%, 0.25-0.7%, or 0.65-0.85% by weight; and a calcium salt may be included up to 0.5%, 0.07-0.12%, 0.01-0.12%, 0.1-1.5%, 0.005-0.12%, 0.01-0.2%, or 0.001-1.5% by weight.

In an embodiment, the prepared personal care product may further comprise a magnesium salt, for example, but not limited to, at up to 0.5%, 0.001-1.5%, 0.03-0.07%, 0.01-0.15%, 0.3-0.7%, and 0.3-1.0% by weight; and/or a manganese salt, for example, at up to 0.5%, up to 0.002%, 0.001-0.002%, 0.0001-0.001%, 0.002-0.01% by weight.

In an embodiment, ions may be added as chloride or other salts to the prepared personal care products. For example, the potassium salt may be potassium chloride (KCl), potassium bicarbonate (KHCO₃), potassium gluconate (C₆H₁₁KO₇), potassium bromide (KBr), potassium fluoride (KF), or potassium iodide (KI); similar anions could also be used with sodium, calcium, magnesium, or other cations.

In an embodiment, a prepared personal care product may have from 0.03% to 0.07% of the magnesium salt, from 0.15% to 0.35% of the potassium salt, from 0.07% to 0.12% of the calcium salt, and from 0.30% to 1.0% of the sodium salt.

In an embodiment, a prepared personal care product can be isotonic with human vaginal fluid. For example, the prepared personal care product can have an osmolality from 100-500 mOsm/kg. The composition for prepared personal care product may be non-toxic and not harmful to the human vagina, not causing excessive cell death or sloughing, not compromising epithelial integrity or disturbing intercellular junctions.

In an embodiment, a prepared personal care product may comprise from 0.15% to 0.35% of the potassium salt, from 0.01% to 0.12% of the calcium salt, and from 0.30% to 1.0% of the sodium salt. Embodiments of the prepared personal care product may be a gel with a pH from 2.9 to 4.6. The composition for personal care products may include up to 2% by weight of lactic acid.

In an embodiment, a prepared personal care product may comprise a ratio of ions that is bio-matched to that of a healthy vaginal biome. Maintaining a bio-matched acidic pH, proper lactic acid concentrations, and bio-matched concentrations of osmotically active solutes allows healthy vaginal bacteria, such as Lactobacillus spp., to prosper and prevents overgrowth of pathogenic bacteria. There is extensive epidemiological evidence that vaginal lactobacilli play an essential role in a person's reproductive health. A lactobacillus concentration of 108 colony-forming units per milliliter of vaginal fluid (cfu/mL) is considered healthy, whereas a concentration of 105 cfu/mL (one thousand times lower) is typical of women with bacterial vaginosis.

In an embodiment, a prepared personal care product may be bio-matched to both a ˜2-to-1 ratio of sodium to potassium and to a pH range of 3.6-4.2, 2.9-3.7 or 3.8-4.6 to promote optimal concentration of lactobacilli. The sodium to potassium ratio may also be, for example, 2:1, 2.5:1, 2.7:1, 3:1, or in the range of 1:1 to 3:1.

A method of manufacturing a vaginal product includes adding the composition for personal care products (compositions A, B, or C), a plurality of salts and an acid, to a solvent, wherein the plurality of salts comprises up to 0.5% by weight of a potassium salt, up to 1.5% by weight of a sodium salt, and up to 0.5% by weight of a calcium salt, so that the lubricant has a pH from 3.0 to 5.0, and an osmolality from 100 to 500 mOsm/kg. The method may further include adding up to 0.5% by weight of a magnesium salt. The solvent may be water. In an embodiment, the potassium salt is potassium chloride (KCl), the sodium salt is sodium chloride (NaCl), the calcium salt is calcium chloride (CaCl₂)), and the magnesium salt is magnesium chloride (MgCl₂). In addition, racemic lactic acid may be added in an amount of from 0.01% to 2.5% by weight.

The potassium salt may be added in an amount of 0.15% to 0.35%, the sodium salt may be added in an amount from 0.15% to 0.35%, the calcium salt may be added in an amount from 0.01% to 0.12%, and the magnesium salt may be added in an amount from 0.30% to 1.0%. The ingredients may be added to achieve an osmolality from 300 to 450 mOsm/kg. The substance may be iso-osmolal with human vaginal fluid.

In an embodiment, the plurality of salts includes up to 0.5% by weight of a potassium salt, up to 1.5% by weight of a sodium salt, and up to 0.5% by weight of a calcium salt. The salts may further include up to 0.5% by weight of magnesium salt and/or up to 0.1% by weight of manganese salt. In an embodiment, the substance has from 0.03% to 0.07% of the magnesium salt, from 0.001 to 0.002% of the manganese salt; from 0.15% to 0.35% of the potassium salt, from 0.01% to 0.12% of the calcium salt and from 0.30% to 1.0% of the sodium salt. The substance may further comprise 0.002-0.008% by weight of sorbic acid, or from 0.001 to 0.01% by weight of sorbic acid.

In an embodiment, a topical gel for human use is provided. The gel may comprise a formulation matched to a composition of a particular part of a human body and the composition for personal care products (compositions A, B, or C). The formulation may include lactic acid, and the particular part may be a vagina or vulva.

In an embodiment, a topical gel for human use may comprise a formulation including lactic acid having a racemic index in a range of about 50% L/50% D and the composition for personal care products (compositions A, B, or C). The formulation may be matched to a composition (or chemistry thereof) of a particular part of a human body.

In an embodiment a topical pain/itch gel for human use may comprise a formulation including bio-matched ion ratios and a pain reliever such as lidocaine (up to 4%) and the composition for personal care products (compositions A, B, or C).

In an embodiment, a vaginal lubricant is provided. The lubricant may comprise a formulation including lactic acid having a racemic index that is bio-matched to a racemic index of natural lubricants in a generally healthy vagina and the composition for personal care products (compositions A, B, or C).

### The Effects and Use of Acemannan

It is appreciated that the use of acemannan has multiple beneficial effects when used in a composition for personal products. First, acemannan provides unexpectedly high viscosity to the composition when combined with succinoglycan, brassicyl valinate esylate, and cetearyl alcohol. It is appreciated that the amount of acemannan needed to achieve high viscosity in combination with succinoglycan, brassicyl valinate esylate, and cetearyl alcohol is significantly lower than the amount needed to achieve the same viscosity by acemannan alone, as discussed below.

Second, unlike crude Aloe vera juice or extract, purified acemannan does not have adverse effects on subjects that a personal product containing acemannan is applied to. Acemannan is a purified form of polymannose (a D-isomer mucopolysaccharide) present in the inner leaf gel of Aloe vera plantis, a medicinal plant. Although Aloe vera is widely used to treat various conditions in humans, some people develop adverse reactions to components present in the plant, and thus using aloe juice or aloe extract is better avoided. The compositions B and C for personal care products comprises purified acemannan. As compositions B and C do not contain other components included in the aloe plant, it has beneficial effects of acemannan, such as providing viscosity to the composition and promoting growth of Lactobacilli but is safer to use in personal products than products containing aloe juice or aloe extract. In addition, it is appreciated that the amount of acemannan in the composition for personal products is lower than the amount of acemannan required to achieve the same viscosity with acemannan alone. Thus, any adverse effect of acemannan, if any, can be reduced.

Third, it is further appreciated that acemannan has prebiotic effects for the growth of Lactobacilli, as described below. The Lactobacilli may be Lactobacillus crispatus or Lactobacillus gasseri which are species of bacteria beneficial for vaginal health. Thus, when used in a vaginal care product, the composition B containing acemannan can promote the growth of beneficial vaginal microbe. The acemannan can be an irradiated form. It is appreciated that the prebiotic effects of acemannan is not limited to Lactobacillus crispatus or Lactobacillus gasseri, and the growth of other Lactobacilli may be enhanced. It is also appreciated that acemannan may be included in personal products other than vaginal care products and have prebiotic effect

### Method of Making a Bio-Matched Personal Product

“Bio-matching” is the method of formulating a product to have similar components as those found in the part of the body for which the product is intended for use. A method for bio-matching is described, for example, in U.S. Pat. Nos. 9,470,676 and 10,195,169 and incorporated herein.

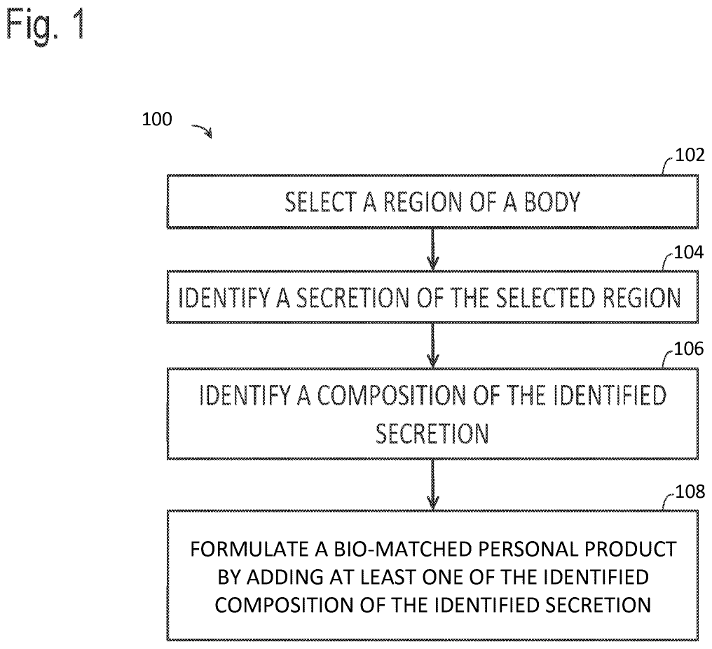

FIG. 1 shows an exemplary method, generally indicated at 100, of bio-matching a composition for personal product to a living body so as to prepare a bio-matched personal product. The bio-matched personal product can be, for example, an ointment, a topical gel, a cream, a lubricant, a wash, a wipe, a serum, a hydrogel, a pain/itch gel, balms, tonics, oils, salves, or other suitable substance. The method 100 starts at 102.

At 102, a region of a body is selected. The selecting may involve selecting a region including one or more components, for example, but not limited to one or more glands, one or more organs, and/or flora (e.g., microflora including bacteria) that secrete various substances. Typically, portions (or regions) of male and female human bodies secrete various natural substances (or secretions). For example, a portion of a female human body or a male human body (e.g., a gland, organ, or flora associated with the portion or an organ) may secrete or produce one or more particular substances (e.g., lactic acid, saliva, etc.) for one or more particular functions (e.g., lubricating, moisturizing, cell protection, cell repair, protection from pathogens or foreign matter such as dust, etc.). Identifying a secretion of the selected region comprises identifying one or more of the secretions. The selected body may be a human body, and the selected region may be a vagina. The method 100 then proceeds to 104.

At 104, a secretion of the selected region is identified. The method at 104 may involve selecting at least one secretion produced by (or present in) a generally healthy region, such as a generally healthy vagina. The 104 may involve identifying a secretion that a relatively healthy body (or part thereof) produces. In an embodiment, 104 may involve identifying salts, electrolytes, other molecules, osmolality, or pH. For example, microflora of a generally healthy vagina typically produces lactic acid comprising approximately 50% L-lactic acid (i.e., L-enantiomers) and 50% D-lactic acid (i.e., D-enantiomers). In contrast, bacterial vaginosis more commonly occurs in vaginas with microflora that produce lactic acid with little or no percentage of D-lactic acid. Accordingly, 104 may involve identifying lactic acid having L-enantiomers and D-enantiomers. The method 100 then proceeds to 106.

At 106, the composition of the identified secretion (e.g., chemical composition, molecular composition, ionic composition, or characteristics or properties thereof) is identified. Identifying the composition of the identified secretion may comprise testing for the presence and/or amounts of various components. In an embodiment, in the case of vagina, a vaginal fluid may be tested for the pH, the sodium concentration, the potassium concentration, the sodium: potassium ratio, the total lactic acid level, the D-lactic acid level, the L-lactic acid level, the presence, amounts, and/or concentrations of various bacteria, moisture level, inflammatory markers, and the presence and/or abundance of immune cells. The method 100 then proceeds to 108.

At 108, method 100 may include selecting at least one of the identified composition of the identified secretion and adding to the composition for personal product so as to obtain a bio-matched personal product. The 108 may involve selecting salts or other electrolytes. The 108 may involve selecting lactic acid that is approximately racemic. The 108 may involve selecting synthetic acid. The 108 may involve selecting racemic synthetic acid. The 108 may involve selecting pure racemic synthetic acid having a racemic index of 50% L/50% D. The 108 may involve formulating the topical gel to include about 1% lactic acid (synthetic and/or racemic). In some embodiments, the 108 may involve bio-matching the topical gel to avoid (or by avoiding) inclusion of one or more ingredients that are toxic or harmful to microbiota of the generally healthy vagina or any vagina. Examples of ingredients that are toxic or harmful to the microbiota of a vagina include detergents, surface-active agents, glycerol, many types of preservatives including EDTA, ion concentrations/formulations that make the formulation non-isotonic in the vagina, and acid/base concentrations/formulations that do not match the pH of the generally healthy vagina. The method 100 ends at 108.

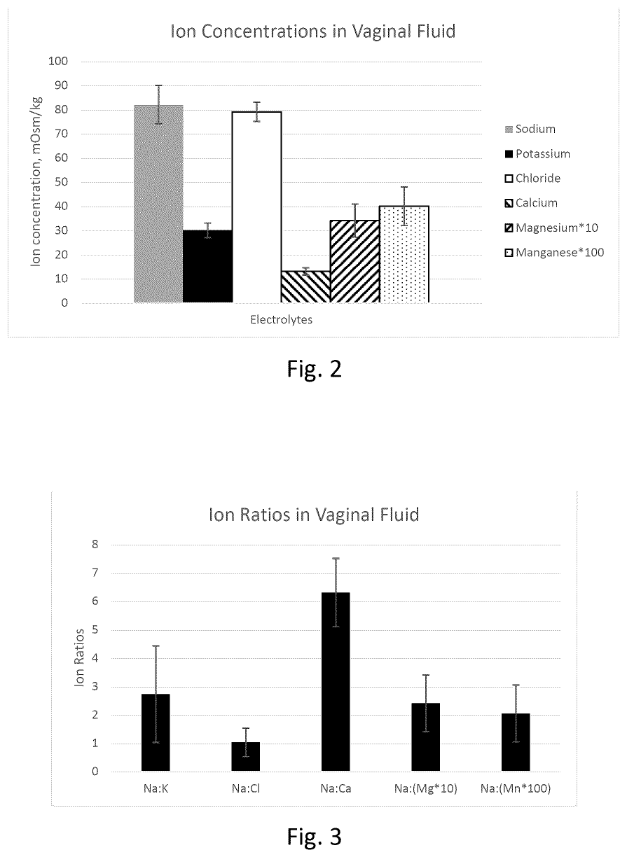

FIG. 2 shows the concentrations of various ions in vaginal fluid according to Mende et al., Zentralbl Gynakol. 1990; 112 (18): 1175-1180. Sodium, potassium, chloride, and calcium are shown as mOsm/kg. Magnesium is shown as 10-times mOsm/kg and manganese is shown as 100 times mOsm/kg for easier interpretation of the graph. In an embodiment, the shown amounts of ions may be combined with the composition for personal products to prepare a vaginal product that is bio-matched.

FIG. 3 shows ratios of sodium to other ions in vaginal fluid, calculated from disclosures in literatures. Other ions include potassium, chloride, calcium, magnesium, and manganese. Magnesium is shown as sodium to 10-times magnesium, and manganese is shown as sodium to 10-times manganese for ease of graph interpretation. In an embodiment, the shown amounts of ions may be combined with the composition for personal products to prepare a vaginal product that is bio-matched.

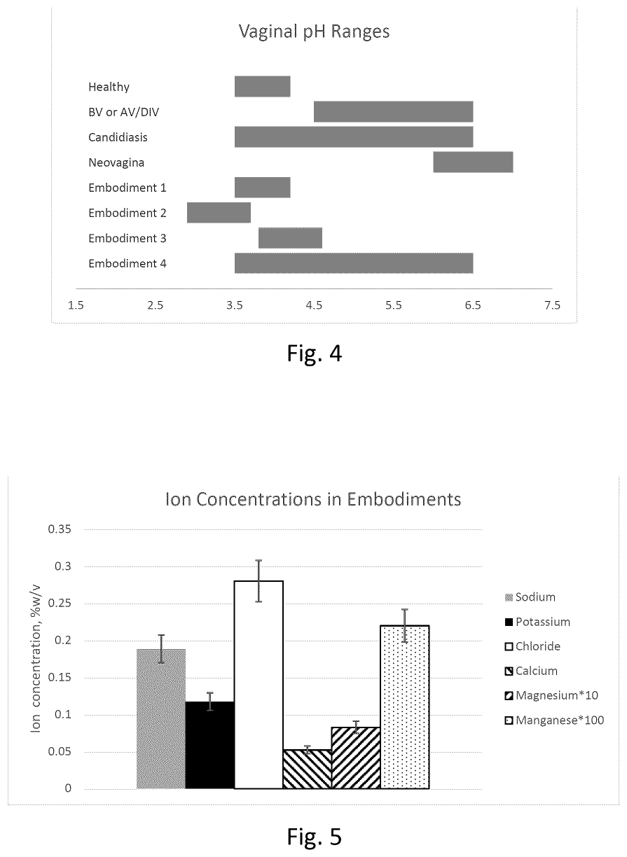

FIG. 4 shows the pH of vaginal fluid in various conditions and in some embodiments. Conditions include healthy, bacterial vaginosis (BV), aerobic vaginitis (AV)/desquamative inflammatory vaginitis (DIV), and vulvovaginal candidiasis (yeast infection). The pH of a neovagina without any intervention is also shown. Vaginal pH is largely reflective of the state of the vaginal microbiome, and in particular, which bacterial species are present in the highest amounts. Native vaginal lactobacilli acidify vaginal fluid more strongly (lower pH) than pathogenic bacteria or non-native lactobacilli. Thus, when native lactobacilli are present—a heathy state—the vaginal pH is approximately 3.5-4.5. During BV and AV/DIV, pathogenic bacteria are present at considerable amounts, and lactobacilli are present in low amounts; this elevates vaginal pH to an unhealthy level: at least 4.5, and sometimes as high as 6.5. Candida yeast species may thrive at any vaginal pH, but over time they tend to raise the pH of vaginal fluid. So, they may be present at a “healthy” pH of 3.5 up to an elevated pH of up to 6.5. Neovaginas often lack lactobacilli because they are derived from scrotal, penile, or other tissues, which do not support lactobacilli well. Thus, neovaginas may have a pH in the range of 6.0 to 7.0. Embodiments of the composition for personal products may have a pH matching that of healthy vaginal fluid, or a pH lower than vaginal fluid to correct vaginal fluid with a pH that is too high, or embodiments may have a pH higher than vaginal fluid to correct vaginal fluid with a pH that is too low, or an embodiment may have a pH that is adjustable based on the presence of other fluids in the vagina.

FIG. 5 shows the concentrations of various ions in an embodiment. Sodium, potassium, chloride, and calcium are shown as % w/v. Magnesium is shown as 10 times % w/v and manganese is shown as 100 times % w/v for easier interpretation of the graph. The ranges shown here are for maintaining a generally healthy vaginal environment. In an embodiment, the shown amounts of ions may be combined with the composition for personal products to prepare a vaginal product that is bio-matched.

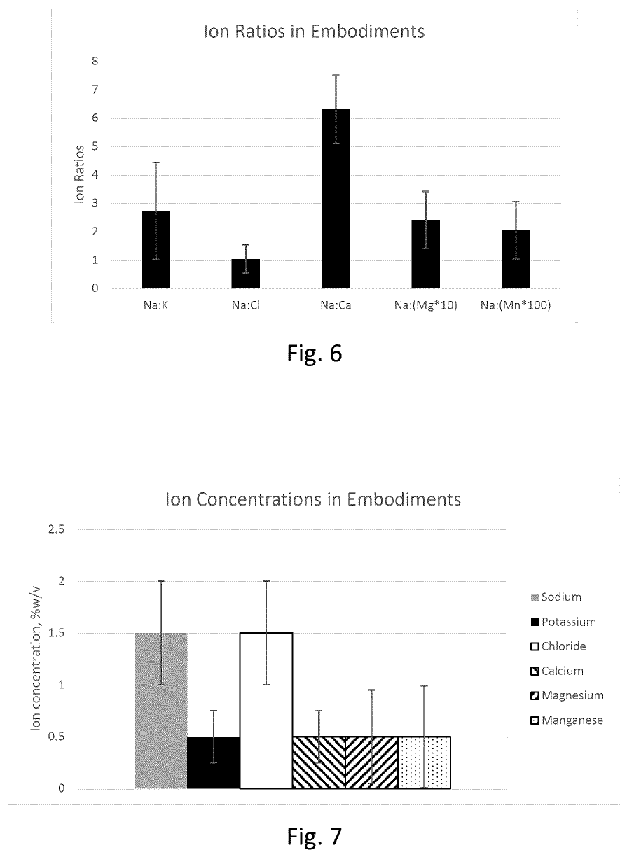

FIG. 6 shows ratios of sodium to other ions in some embodiments. Other ions include potassium, chloride, calcium, magnesium, and manganese. Magnesium is shown as sodium to 10-times magnesium, and manganese is shown as sodium to 10-time manganese for ease of graph interpretation. In an embodiment, the ions may be combined at the shown ratios with the composition for personal products to prepare a vaginal product that is bio-matched.

FIG. 7 shows possible ranges of ion concentrations in an embodiment comprising sodium, potassium, chloride, calcium, magnesium, and optionally manganese. Concentrations are shown as % w/v. The ranges shown here are an example of ion concentrations for treating patients having low levels of sodium, potassium, chloride, calcium, magnesium, and/or manganese due to a vaginal dysbiosis or other vaginal condition. In an embodiment, the shown amounts of ions may be combined with the composition for personal products to prepare a vaginal product that is bio-matched.

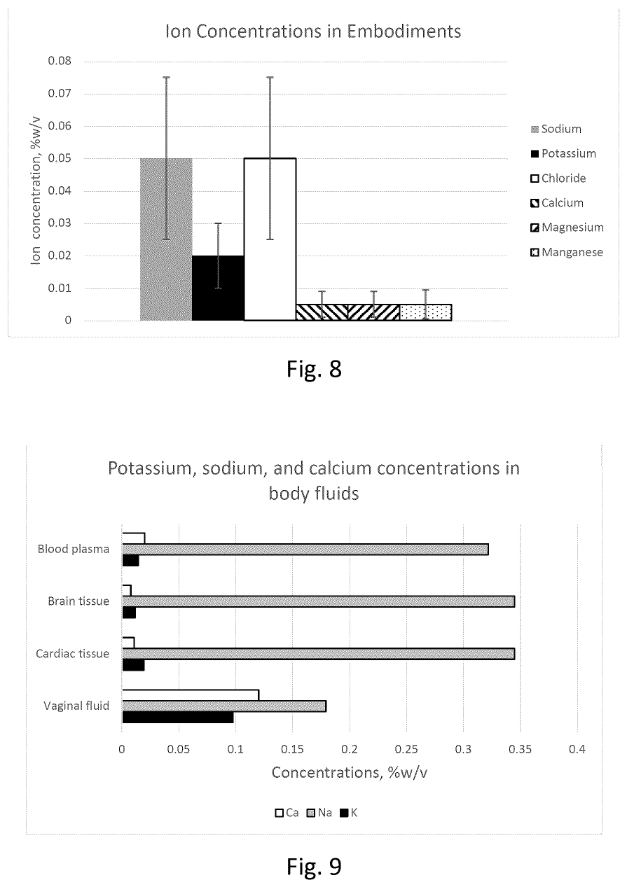

FIG. 8 shows possible ranges of ion concentrations in an embodiment for sodium, potassium, chloride, calcium, magnesium, and optionally manganese. Concentrations are shown as % w/v. The ranges shown here are an example of ion concentrations for treating patients having high levels of sodium, potassium, chloride, calcium, magnesium, and/or manganese due to a vaginal dysbiosis or other vaginal condition. In an embodiment, the shown amounts of ions may be combined with the composition for personal products to prepare a vaginal product that is bio-matched.

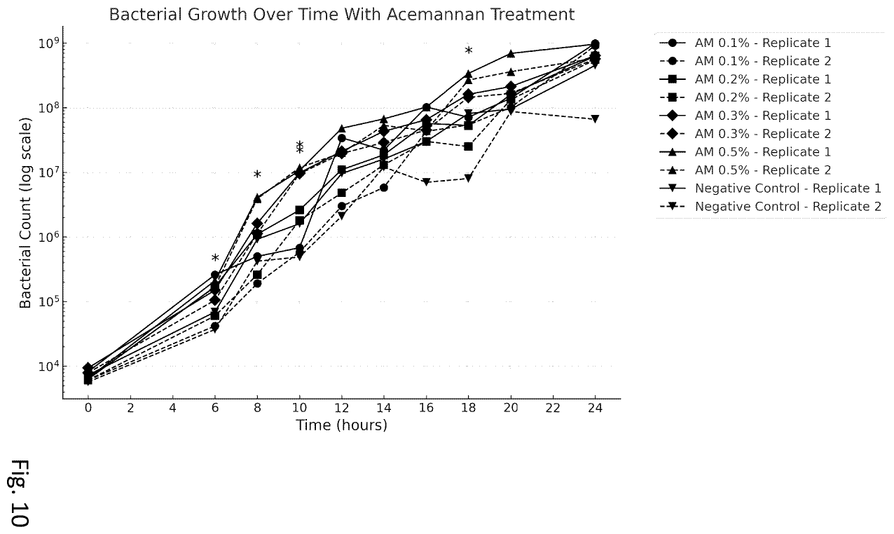

FIG. 9 shows sodium, potassium, and calcium concentrations in body fluids according to literatures. As can be seen in FIG. 9, the variation of the amounts of calcium, sodium and potassium amounts are moderate among blood, brain, and heart; however, the ion concentrations differ significantly in the female vagina. This distinct composition is necessary for the survival of sperm cells, for the survival and function of epithelial cells and for the maintenance of a healthy vaginal microbiome. These data indicate that topical compositions designed for generic use or other parts of the body are not suited as vaginal compositions and show the need and benefit for preparing a product that is bio-matched to a healthy vaginal fluid.

### EXAMPLES

### Example 1

The following describes a study to determine the viscosity of a composition containing succinoglycan, brassicyl valinate esylate, brassica alcohol, and brassica glycerides.

RHEOZAN® SH available from SYENSQO was used as succinoglycan. AMINOSENSYL™ SC is available from INOLEX and is a mixture of brassicyl valinate esylate, brassica alcohol, and brassica glycerides. Examples 1-1 to 1-4 were prepared according to Table 1 below. Compositions containing varying ratios of RHEOZAN® SH and AMINOSENSYL™ SC were prepared in water and mixed until homogenized at 70° C. The contents were stirred until fully dissolved and cooled to 25° C. The viscosity of the compositions was measured with a NDJ5S rotational viscometer (Shandong Drick Instruments) with a #4 spindle at 6 rpm.

TABLE 1

| | Exam- | Exam- | Exam- | Exam- |
|---|---|---|---|---|
| weight (%) | ple 1-1 | ple 1-2 | ple 1-3 | ple 1-4 |
| RHEOZAN ® SH | 1.0% | 1.0% | 1.0% | 0% |
| AMINOSENSYL ™ SC | 0% | 0.25% | 2.00% | 1.0% |
| Viscosity (mPaS) | 44320 | 73200 | 23187 | 932 |

Results show that a solution containing RHEOZAN® SH at about 1% and AMINOSENSYL™ SC at about 0.25% has unexpectedly high viscosity compared with solutions containing RHEOZAN® SH alone or AMINOSENSYL™ SC alone.

### Example 2

The following describes a study to determine the viscosity of a solution containing succinoglycan, brassicyl valinate esylate, brassica alcohol, brassica glycerides and acemannan.

Examples 2-1 and 2-2 were prepared as below, according to Table 2. The indicated amount of water was added to a vessel and stirred. The pH was adjusted with sodium hydroxide to be higher than 9, and acemannan was added slowly to the water to dissolve. Next RHEOZAN® SH was added slowly while continuously stirring the solution. The vessel was heated until the content reach 70° C. and then allowed to cool to 45° C. Potassium Sorbate and Sodium Benzoate were then added and stirred until fully dissolved. Lactic acid was added to adjust the pH of the product to 3.7 to 4. The product was cooled to 25° C. and viscosity was measured as described above.

Examples 2-3 and 2-4 were prepared according to Table 2, with the same steps as Examples 2-1 and 2-2; however, the product became very viscous and did not form a homogenous gel. The viscosity could not be measured by the method above because the products essentially solidified.

Example 2-5 was prepared according to Table 2, with the same steps as Examples 2-1 and 2-2, except after adding RHEOZAN SH, pellets of AMINOSENSYL™ SC were slowly added while stirring the solution. The viscosity was measured as described above.

Example 2-6 was prepared according to Table 2, with the same steps as Example 2-5 except AMINOSENSYL™ SC was not added to the solution.

TABLE 2

| | Example 2-1 | | Example 2-2 | | Example 2-3 | | Example 2-4 | | Example 2-5 | | Example 2-6 | |
|---|---|---|---|---|---|---|---|---|---|---|---|---|
| | weight | weight | weight | weight | weight | weight | weight | weight | weight | weight | weight | weight |
| | (g) | (%) | (g) | (%) | (g) | (%) | (g) | (%) | (g) | (%) | (g) | (%) |
| Acemannan | 0.21 | 0.1% | 0.66 | 0.3% | 1.1 | 0.5% | 2.2 | 1.0% | 0.22 | 0.1% | 0.66 | 0.3% |
| Potassium | 0.71 | 0.3% | 0.7 | 0.3% | 0.7 | 0.3% | 0.7 | 0.3% | 0.71 | 0.3% | | |
| Sorbate | | | | | | | | | | | | |
| Deionized | 213.7 | 97.5% | 213.2 | 97.3% | 213.3 | 97.1% | 212.2 | 96.6% | 214 | 97.4% | 200 | 99.3% |
| water | | | | | | | | | | | | |
| Rheozan SH | 2.2 | 1.0% | 2.2 | 1.0% | 2.1 | 1.0% | 2.1 | 1.0% | 2.2 | 1.0% | | |
| D/L Lactic | 1.72 | 0.8% | 1.7 | 0.8% | 1.7 | 0.8% | 1.7 | 0.8% | 1.71 | 0.8% | 0.7 | 0.3% |
| acid | | | | | | | | | | | | |
| Na Benzoate | 0.7 | 0.3% | 0.7 | 0.3% | 0.7 | 0.3% | 0.7 | 0.3% | 0.7 | 0.3% | | |
| Sodium | 0.01 | 0.005% | 0.01 | 0.005% | 0.01 | 0.005% | 0.01 | 0.005% | 0.01 | 0.005% | 0.01 | 0.005% |
| Hydroxide | | | | | | | | | | | | |
| Aminosensyl | | | | | | | | | 0.21 | 0.1% | | |

| | | | | | | |
|---|---|---|---|---|---|---|
| pH | 3.7-4 | 3.7-4 | 3.7-4 | 3.7-4 | 3.7-4 | 3.9 |
| Viscosity | 62137 | 95231 | *gelled | *gelled | 87212 | 27312 |
| (mPaS) | | | | | | |

The results show that combining about 0.1-0.3% of Acemannan with about 1% of RHEOZAN SH unexpectedly increases the viscosity compared with RHEOZAN alone or acemannan alone (compare Examples 2-1 and 2-2 with Examples 1-1 and 2-6). The compositions each formed stable mixtures at pH 3.7 to 4.0. The results also show that adding about 0.1% of AMINOSENSYL™ SC to the Acemannan-RHEOZAN® SH solution significantly increases the viscosity of the product (compare Examples 2-1 and 2-5).

### Example 3

The following describes a study to determine the viscosity of compositions containing different amounts of succinoglycan, brassicyl valinate esylate, brassica alcohol, brassica glycerides and acemannan.

Examples 3-1 to 3-8 were prepared according to Table 3, according to the same protocol as described above for Examples 2-1 and 2-2, except the amounts of RHEOZAN® SH were varied in Examples 3-1 to 3-4, the amounts of acemannan were varied in Examples 3-5 and 3-6, and the amounts of AMINOSENSYL™ SC in Examples 3-7 and 3-8. The viscosity was measured as described above. The osmolality was measured by an osmometer (Model 3320) from Advanced Instruments Inc.

The results show that the combinations comprising RHEOZAN® SH, AMINOSENSYL™ SC, and acemannan has unexpectedly high viscosity compared with each component alone. The compositions each formed stable mixtures at pH 3.7 to 4.0.

TABLE 3

| | Example 3-1 | | Example 3-2 | | Example 3-3 | | Example 3-4 | |
|---|---|---|---|---|---|---|---|---|
| | weight | weight | weight | weight | weight | weight | weight | weight |
| | (g) | (%) | (g) | (%) | (g) | (%) | (g) | (%) |
| Acemannan | 0.54 | 0.20 | 0.20 | 0.20 | 0.55 | 0.20 | 0.55 | 0.19 |
| Potassium | 0.70 | 0.25 | 0.25 | 0.25 | 0.74 | 0.27 | 0.73 | 0.25 |
| Sorbate | | | | | | | | |
| Deionized | 269.00 | 97.67 | 97.70 | 97.65 | 279.50 | 97.61 | 279.50 | 97.51 |
| water | | | | | | | | |
| Rheozan SH | 1.08 | 0.39 | 0.40 | 0.40 | 1.35 | 0.49 | 1.65 | 0.58 |
| D/L Lactic | 1.65 | 0.60 | 0.60 | 0.60 | 1.72 | 0.62 | 1.72 | 0.60 |
| acid | | | | | | | | |
| Na Benzoate | 0.54 | 0.20 | 0.20 | 0.20 | 0.55 | 0.20 | 0.55 | 0.19 |
| Sodium | qs | qs | 0.001 | 0.001 | qs | qs | qs | qs |
| Hydroxide | | | | | | | | |
| Aminosensyl | 0.27 | 0.10 | 0.10 | 0.10 | 0.28 | 0.10 | 0.28 | 0.10 |
| Natrasol | 1.64 | 0.60 | 0.60 | 0.60 | 1.65 | 0.60 | 1.66 | 0.58 |
| 250 HHR | | | | | | | | |
| pH | 3.75 | | 3.73 | | 3.91 | | 3.91 | |
| Viscosity | 38009 | | 38271 | | 43212 | | 52181 | |
| (mPas) | | | | | | | | |
| osmolality | 307 | | 309 | | 321 | | 311 | |
| | Example 3-5 | | Example 3-6 | | Example 3-7 | | Example 3-8 | |
| | weight | weight | weight | weight | weight | weight | weight | weight |
| | (g) | (%) | (g) | (%) | (g) | (%) | (g) | (%) |
| Acemannan | 0.40 | 0.40 | 0.50 | 0.50 | 0.20 | 0.20 | 0.20 | 0.20 |
| Potassium | 0.25 | 0.25 | 0.25 | 0.25 | 0.25 | 0.25 | 0.25 | 0.25 |
| Sorbate | | | | | | | | |
| Deionized | 97.50 | 97.45 | 97.40 | 97.35 | 97.90 | 97.70 | 97.45 | 97.45 |
| water | | | | | | | | |
| Rheozan SH | 0.40 | 0.40 | 0.40 | 0.40 | 0.40 | 0.40 | 0.40 | 0.40 |
| D/L Lactic | 0.60 | 0.60 | 0.60 | 0.60 | 0.60 | 0.60 | 0.60 | 0.60 |
| acid | | | | | | | | |
| Na Benzoate | 0.20 | 0.20 | 0.20 | 0.20 | 0.20 | 0.20 | 0.20 | 0.20 |
| Sodium | 0.001 | 0.001 | 0.001 | 0.001 | 0.001 | 0.001 | 0.001 | 0.001 |
| Hydroxide | | | | | | | | |
| Aminosensyl | 0.10 | 0.10 | 0.10 | 0.10 | 0.05 | 0.05 | 0.30 | 0.30 |
| Natrasol | 0.60 | 0.60 | 0.60 | 0.60 | 0.60 | 0.60 | 0.60 | 0.60 |
| 250 HHR | | | | | | | | |

| | | | | |
|---|---|---|---|---|
| pH | 3.73 | 3.71 | 3.81 | 3.91 |
| Viscosity | 51718 | 53211 | 19541 | 52181 |
| (mPas) | | | | |
| osmolality | 307 | 302 | 311 | 311 |

### Example 4

The following describes a study to determine the effect of Aloe vera extract containing acemannan on the growth of Lactobacillus Crispatus and Lactobacillus gasseri. Different prebiotics and/or different organisms may be tested by this method. These studies are conducted to determine whether components in the Aloe vera extract negatively or positively impacts the growth of lactobacillus strains. In addition, the pH of cultures is measured. A culture without the test prebiotic serves as the negative control.

### Methods

Irradiated Aloe vera inner leaf extract (NaturManan® Professional) at 0.1% and 0.3% w/v was added to the Lactobacillus crispatus and Lactobacillus gasseri culture tubes prepared in dextrose-free MRS broth. Cultures without the aloe extract was prepared as control.

Growth media contained Dextrose-Free Sharpe (MRS) broth and Lactobacilli MRS Agar or an equivalent. Extract of irradiated aloe vera was further added to the MRS broth at 0.1 and 0.3% (w/v). A predetermined number of bacteria was added to each of the prepared medium and cultured at 37° C. under a suitable condition for the organism to be tested (anaerobic conditions for L. crispatus and L. gasseri). A small portion of the culture was removed at 2, 4, 6, 8, 12, 14, 16, 18, and 24 hours of culture. At each time point, collected samples containing the growing organisms are plated to quantify the live cell number.

The cell growth of Lactobacillus crispatus and Lactobacillus gasseri was determined by serial dilutions and plating to determine the bacterial counts at designated time points during the 24-h study period. Plates were cultured at 37° C. under a suitable condition for the organism to be tested (anaerobic conditions for L. crispatus and L. gasseri). Bacterial colonies were quantified at 0 and 16 hours after incubation.

### Results

L. Crispatus cultured in the presence of 0.1% Aloe vera leaf extract showed five-fold increase in the colony-forming-unit (CFU), and L. Crispatus cultured in the presence of 0.3% Aloe vera leaf extract showed 2.5-fold increase in the CFU at 16 hours of culture compared with control cultured in a medium without Aloe vera leaf extract.

### Example 5

The following describes a study to determine the effect of acemannan on the growth of Lactobacillus Crispatus. Different prebiotics and/or different organisms may be tested by this method. These studies are conducted to determine whether acemannan has prebiotic effects on the growth of Lactobacillus Crispatus. In addition, the pH of cultures is measured. A culture without the test prebiotic serves as the negative control.

### Methods

The experiments were carried out using the method described above for EXAMPLE 4. Lactobacillus crispatus was cultured in the presence of varying concentrations (0.1%, 0.2%, 0.3%, 0.5%) of UV-irradiated acemannan or a negative control over a 24-hour period. Bacterial growth was quantified at 10 time points (0-24 hours) using log-scaled CFU/mL equivalents.

### Results

The results are shown in FIG. 10. Solid lines represent Replicate 1; dashed lines represent Replicate 2. Distinct black-and-white markers were used for each condition to aid visualization. Asterisks (*) indicate time points at which the acemannan-including culture showed statistically significant increases in growth over the negative control (p<0.05, unpaired t-test). The culture containing 0.5% acemannan produced the most robust and consistent stimulation of L. crispatus growth, with significant effects observed at 6, 8, 10, and 18 hours. The culture containing 0.3% acemannan produced significant enhancement of growth at 10 hours compared with negative control.

### ASPECTS

It is noted that any of aspects 1-7 can be combined with any one of aspects 8-14 or aspects 15-18. Any one of aspects 8-14 can be combined with any one of aspects 15-18.

Aspect 1. A composition for personal care products comprising succinoglycan, brassicyl valinate esylate, and cetearyl alcohol.

Aspect 2. The composition according to aspect 1, wherein the composition is acidic.

Aspect 3. The composition according to any one of aspects 1-2, further comprising lactic acid.

Aspect 4. The composition according to any one of aspects 1-3, further comprising electrolytes that match the concentration of electrolytes found in a vaginal fluid.

Aspect 5. The composition according to any one of aspects 1-4, wherein, by a total weight of the composition, an amount of the succinoglycan is 1.0% or lower, and a combined amount of the brassicyl valinate esylate and cetearyl alcohol is 4% or lower.

Aspect 6. The composition according to any one of aspects 1-5, wherein the composition is a fluid, and a viscosity of the composition is at least 30,000 mPaS when measured using a NDJ5S rotational viscometer with a #4 spindle at 6 rpm.

Aspect 7. The composition according to any one of aspects 1-6, wherein a change rate of the viscosity of the composition after storing at 40° C. for 40 weeks is less than 10%.

Aspect 8. A composition for personal care products comprising succinoglycan, brassicyl valinate esylate, cetearyl alcohol and acemannan.

Aspect 9. The composition according to aspect 8, wherein the composition is acidic.

Aspect 10. The composition according to any one of aspects 8-9, further comprising lactic acid.

Aspect 11. The composition according to any one of aspects 8-10, further comprising electrolytes that match the concentration of electrolytes found in a vaginal fluid.

Aspect 12. The composition according to any one of aspects 8-11, wherein, by a total weight of the composition, an amount of the succinoglycan is 1.0% or lower, a combined amount of the brassicyl valinate esylate and cetearyl alcohol is 4% or lower, and an amount of the acemannan is 2% or lower.

Aspect 13. The composition according to any one of aspects 8-12, wherein the composition is a fluid, and a viscosity of the composition is at least 30,000 mPaS when measured using a NDJ5S rotational viscometer with a #4 spindle at 6 rpm.

Aspect 14. The composition according to any one of aspects 8-13, wherein a change rate of the viscosity of the composition after storing at 40° C. for 40 weeks is less than 10%.

Aspect 15. A composition for personal care products comprising acemannan, wherein the composition has a prebiotic effect for vaginal bacteria.

Aspect 16. The composition according to aspect 15, wherein the prebiotic effect includes enhancement of proliferation of Lactobacilli.

Aspect 17. The composition according to any one of aspects 15-16, wherein the Lactobacilli is selected from the group consisting of Lactobacillus crispatus and Lactobacillus gasseri.

Aspect 18. The composition of according to any one of aspects 15-17, wherein the acemannan is irradiated.

The disclosure set forth herein encompasses multiple distinct embodiments with independent utility. These embodiments are not to be considered in a limiting sense as numerous variations are possible. Each example defines an embodiment disclosed in the foregoing disclosure, but any one example does not necessarily encompass all features or combinations that may be eventually claimed. Where the description recites “a” or “a first” element or the equivalent thereof, such description includes one or more such elements, neither requiring nor excluding two or more such elements. Further, ordinal indicators, such as first, second or third, for identified elements are used to distinguish between the elements, and do not indicate a required or limited number of such elements, and do not indicate a particular position or order of such elements unless otherwise specifically stated.

1. A composition for personal care products comprising succinoglycan, brassicyl valinate esylate, and cetearyl alcohol.

2. The composition of claim 1, wherein the composition is acidic.

3. The composition of claim 1, further comprising lactic acid.

4. The composition of claim 1, further comprising electrolytes that match the concentration of electrolytes found in a vaginal fluid.

5. The composition of claim 1, wherein, by a total weight of the composition, an amount of the succinoglycan is 1.0% or lower, and a combined amount of the brassicyl valinate esylate and cetearyl alcohol is 4% or lower.

6. The composition of claim 1, wherein the composition is a fluid, and a viscosity of the composition is at least 30,000 mPaS when measured using a NDJ5S rotational viscometer with a #4 spindle at 6 rpm.

7. The composition of claim 6, wherein a change rate of the viscosity of the composition after storing at 40° C. for 40 weeks is less than 10%.

8. A composition for personal care products comprising succinoglycan, brassicyl valinate esylate, cetearyl alcohol and acemannan.

9. The composition of claim 8, wherein the composition is acidic.

10. The composition of claim 8, further comprising lactic acid.

11. The composition of claim 8, further comprising electrolytes that match the concentration of electrolytes found in a vaginal fluid.

12. The composition of claim 8, wherein, by a total weight of the composition, an amount of the succinoglycan is 1.0% or lower, a combined amount of the brassicyl valinate esylate and cetearyl alcohol is 4% or lower, and an amount of the acemannan is 2% or lower.

13. The composition of claim 8, wherein the composition is a fluid, and a viscosity of the composition is at least 30,000 mPaS when measured using a NDJ5S rotational viscometer with a #4 spindle at 6 rpm.

14. The composition of claim 8, wherein a change rate of the viscosity of the composition after storing at 40° C. for 40 weeks is less than 10%.

15. A composition for personal care products comprising acemannan, wherein the composition has a prebiotic effect for vaginal bacteria.

16. The composition of claim 15, wherein the prebiotic effect includes enhancement of proliferation of Lactobacilli.

17. The composition of claim 16, wherein the Lactobacilli is selected from the group consisting of Lactobacillus crispatus and Lactobacillus gasseri.

18. The composition of claim 15, wherein the acemannan is irradiated.
